(19) United States
(12) Patent Application Publication
(10) Pub. No.: US 2011/0120459 A1
(43) Pub. Date: May 26, 2011

(54) ILLUMINATED ENDOTRACHEAL STYLET

(75) Inventor: Patricia Ramos, Ware, MA (US)

(73) Assignee: OMNIGLOW LLC, Indian Orchard, MA (US)

(21) Appl. No.: 12/622,711

(22) Filed: Nov. 20, 2009

(51) Int. Cl. A61M 16/00; B23P 17/00

(52) U.S. Cl. 128/200.26; 29/454

(57) ABSTRACT

A stylet for use with an endotracheal tube includes a lighting element and a malleable rod completely enclosed and sealed within an elongated flexible sheath. The elongated flexible sheath has a proximal end and a distal end; the lighting element is positioned at or near the distal end and the malleable rod is positioned from proximate to the lighting element to the proximal end of the elongated flexible sheath. The chemiluminescent lighting element includes an activator and an oxalate and may be fully enclosed in a capsule or the flexible sheath may serve as an outer wall of the lighting element. An absorbent pad, made from cotton or non-woven fiber, is at the distal end of the elongated sheath between the lighting element and a terminal seal of the elongated flexible sheath. The stylet includes one or more tactile elements and a removable protective cap.

### BACKGROUND OF THE INVENTION

The present invention is directed to a device to assist in intubation of patients. More particularly, the present invention pertains to a moldable intubation stylet having an illuminated end to facilitate guiding the intubation tube into a cavity or narrow passage.

An endotracheal intubation is a medical procedure in which a tube is placed into the windpipe (trachea) to administer oxygen, medication, or anesthesia. A laryngoscope is generally used to move the tongue out of way and find the trachea. Landmarks, such as the vocal cords, are used to differentiate the trachea from the esophagus, which lie next to one another in the human body. An oxygen tube is inserted into the trachea to provide an open airway. These oxygen tubes are generally flexible or not rigid; thus, a somewhat rigid stylet is often used to provide rigidity to the tube while the tube is being inserted and to provide curvature to the oxygen tube when needed. Once the tube is inserted, the stylet can be disengaged from the oxygen tube and the oxygen tube can be connected to an airway device to supply oxygen to the patient.

Even in the most convenient settings, it is a challenge to intubate patients. Obese patients, for example, often have large tongues and short necks, making it difficult to locate the vocal cords. Small children, likewise, have short necks and small jaws, providing a small workspace and making it difficult to locate the vocal cords.

Facial trauma also provides challenges for intubation. Often the anatomy of the person has changed due to trauma, making it more difficult to locate and open an airway. Large overbites also pose a problem as teeth obstruct light, making it difficult for the physician or emergency personnel to view the pharynx and larynx. In addition, a patient's oral cavity may be filled with fluid which also inhibits correct positioning of the endotracheal tube. Emergency personnel or physicians may attempt several times to intubate a patient. Each attempt adds to the trauma suffered by the patient, as the mouth becomes bruised and sore from the various attempts.

When it is difficult to locate the vocal cords of the trachea, often the esophagus is intubated by accident, causing the patient to regurgitate stomach contents which can flow into the lungs with any subsequent attempt at intubation. This can lead to infection and exacerbation of any underlying trauma. Thus, devices which facilitate lighting of the oral cavity are attractive.

Often the laryngoscopes have lights to illuminate the oral cavity; however, this light is often dim and is often not positioned in a manner that will illuminate a person's throat past, for example, the tonsils. In addition, it is difficult to hold a person's head in the correct position and juggle the laryngoscope, as well as the endotracheal tube and stylet.

Stylets which incorporate lighting into it are useful, but there are many disadvantages to those currently available. For example, bulbs have been known to become disengaged from the stylet and lodge in a patient's throat. Other lighted stylets have relatively dim lights that do not adequately light the trachea or airway. Furthermore, batteries and wiring make lighted stylets cumbersome and unduly complicated.

Accordingly, there is a need for a lighted-type stylet that is easy to use and not unduly cumbersome. Such a stylet, desirably, is self-contained, needing no additional batteries or wiring elements and has sufficient light to illuminate even the darkest cavities.

### BRIEF SUMMARY OF THE INVENTION

A stylet for use with an endotracheal tube includes a lighting element and a malleable rod which are completely enclosed within an elongated flexible sheath. The elongated flexible sheath has a proximal end and a distal end. In one embodiment, the lighting element is positioned at the distal end of the elongated flexible sheath and the malleable rod is positioned from a proximal end of the lighting element to the proximal end of the elongated flexible sheath. In another embodiment, the lighting element is positioned between two malleable rods.

The lighting element is a chemiluminescent lighting element and includes an activator and an oxalate. The oxalate is enclosed within a glass vial separated from the activator. In an embodiment, the two components of the lighting element, the activator and the oxalate, are fully enclosed in a capsule. In another embodiment, the elongated flexible sheath of the stylet serves to encapsulate the lighting element by acting as both the sheath and as an outer wall of the lighting element, eliminating the tube-within-a-tube configuration. The lighting element is separated from the malleable rod by a barrier, such as a hot melt plug or seal.

The malleable rod may be an annealed aluminum rod or a galvanized metal rod and provides both rigidity and necessary flexibility to the stylet. In one embodiment, the lighting element is flanked on both sides by a malleable rod. In another embodiment, the malleable rod is positioned at the proximal end of the stylet while the lighting element is positioned at the distal end.

The elongated flexible sheath encloses the lighting element and the malleable rod(s) and includes a proximal seal at the proximal end and a distal seal at the distal end. The distal seal includes at least one of an absorbent pad and/or a hot melt plug. An absorbent pad, made from cotton or non-woven fiber, is at the distal end of the elongated sheath between the lighting element and a terminal seal of the elongated flexible sheath.

The stylet can include one or more tactile elements configured to enable location of the lighting element or the malleable rod. The tactile element can be a concentric ring, an etching, an embossing, an adhesive-backed emblem, or an otherwise textured area of the flexible sheath.

The stylet can also include a protective cap or sleeve that is removable from the distal end of the stylet. The protective sleeve or cap may be rigid or slightly flexible and can either extend the entire length of the stylet or extend over a portion of the distal end of the stylet. The opaque protective cap can include a lubricant, and prevents bending of the stylet, premature activation of the lighting element, and exposure of the stylet to ultraviolet light.

The lighting element has at least a live (5) minute total effective light output, with a 20-30 second peak light output or brightness. The lighting element may be formulated to glow a variety of different colors, such as pink, red, yellow, blue, green, aqua, purple, and white. It is contemplated that specific colors may be chosen depending on the lighting requirements and specific application conditions. In addition, the lighting element may be configured for temperature variations. For example, it is contemplated that an embodiment of a lighting element formulation may be suitable for warm weather while another embodiment is suitable for cold weather application or both. In yet another embodiment, the light may appear brighter when properly placed in, for example the trachea, and not glow when placed incorrectly, such as in the esophagus. Such an embodiment minimizes false placement of the stylet.

A method of making a chemiluminescent stylet for use with an endotracheal tube includes extruding or providing an elongated, polymer, flexible sheath and heat-sealing the flexible polymer sheath at a first, distal end. The lighting element is added by inserting an oxalate vial and vacuum-filling the flexible polymer sheath with an activator. Tactile elements may be introduced in an embodiment by, for example, inserting a concentric ring into the elongated flexible sheath. A malleable rod is inserted and the proximal end (second end) of the flexible sheath is heat-sealed. The entire sealed stylet is washed in methanol. The malleable rod may be an annealed aluminum rod or a galvanized metal rod. Other tactile elements, such as laser etching may be introduced to the elongated flexible sheath as well.

In another embodiment, a second malleable rod is inserted into the distal end of the elongated flexible sheath, prior to insertion of the lighting elements. In yet another embodiment, as each of the stylet elements is added, it is followed by sealing or a hot plug in order to separate the lighting elements from the malleable rod within the elongated flexible sheath.

### BRIEF DESCRIPTION OF THE SEVERAL VIEWS OF THE DRAWINGS

The benefits and advantages of the present invention will become more readily apparent to those of ordinary skill in the relevant art after reviewing the following detailed description and accompanying drawings, wherein:

FIG. 1 is a side view of a style in accordance with the principles of the present invention;

FIG. 2 is another embodiment of the stylet; and

FIG. 3 is a side view of a lighting element for use with the stylet of FIG. 1.

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiment illustrated.

It should be further understood that the title of this section of this specification, namely, “Detailed Description Of The Invention”, relates to a requirement of the United States Patent Office, and does not imply, nor should be inferred to limit the subject matter disclosed herein.

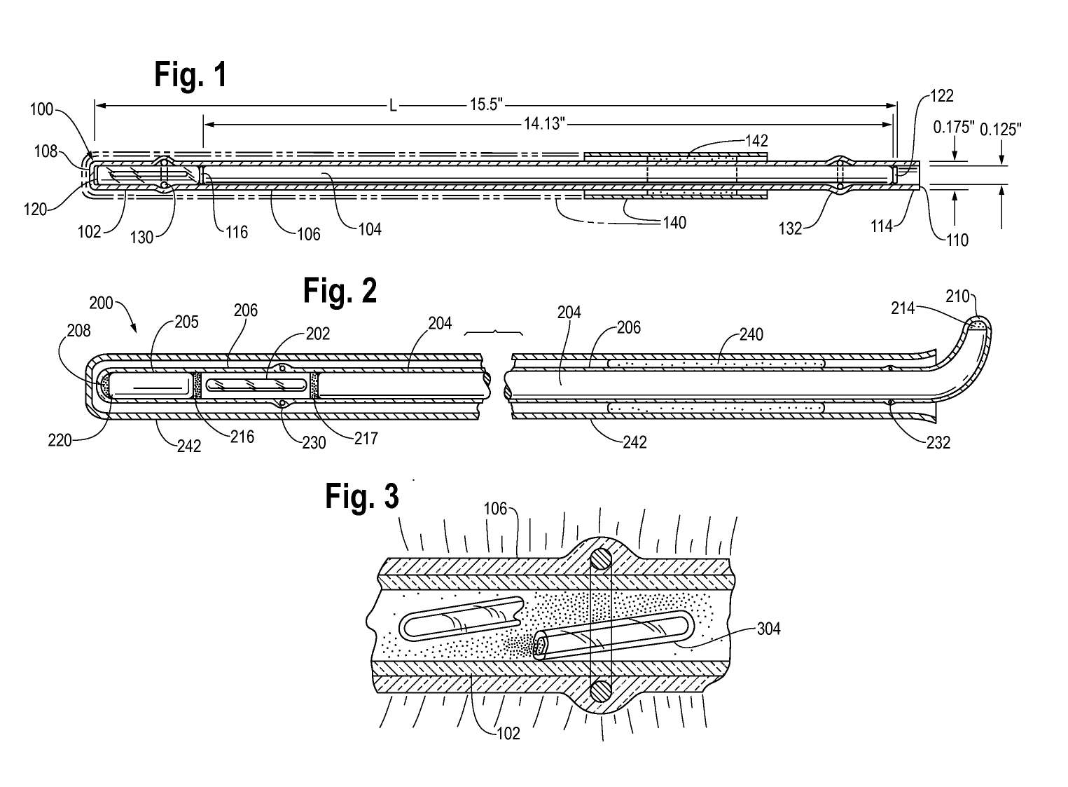

Referring now to FIG. 1, a stylet 100 for use with an endotracheal tube includes a lighting element 102 and a malleable rod 104 completely enclosed within a transparent or translucent, elongated, flexible, polymer sheath 106. The elongated flexible sheath 106 has a proximal end 110 and a distal end 108. In an embodiment, the lighting element 102 is positioned at the distal end 108 of the elongated flexible sheath 106 and the malleable rod 104 is positioned from the proximal end of the lighting element 102 to the proximal end 110 of the elongated flexible sheath 106. In another embodiment of the stylet 200, shown in FIG. 2, the lighting element 202 is positioned between two (2) malleable rods, 204, 205.

The rods 104, 204, 205 may be aluminum alloy, galvanized metal, or other malleable metallic material, or may be made from a flexible polymer. In one example of an embodiment, an outer total length L of the stylet is about 15 inches to about 17 inches. In another example, the outer diameter of the stylet is 0.10-0.20 inches and most preferably about 0.10 inches in diameter. In vet another example of an embodiment, the stylet has an inner diameter of approximately 0.140 inches. It is understood that the dimensions given are for example purposes only and other sizes and shapes of stylets are within the spirit and scope of the present disclosure. The malleable rods 104, 204, 205 provide rigidity to the stylet 100, 200, while also providing needed flexibility. The stylet can be bent or twisted and the malleable rods retain the bent/twisted shape to facilitate insertion into the trachea.

The lighting element 102, 202 is a chemiluminescent lighting element including at least two components. In chemiluminescence, two or more components react to emit light.

In one embodiment, a flexible plastic tube encloses two components, an activator and an oxalate. One of the components, typically the oxalate, is contained within a frangible (typically borosilicate glass) vial within the plastic tube to separate the activator from the oxalate. As shown in FIG. 1, the lighting element 102, 202, with activator and oxalate vial, may be fully enclosed in a plastic tube or capsule 304. In such an embodiment, the lighting element is double enclosed by the plastic tube and by the elongated flexible sheath.

In another embodiment, the elongated flexible sheath 106, 206 itself serves to contain the activator. The flexible plastic tube is eliminated and the sheath of the stylet serves to encase the lighting element components, eliminating the need for a vial within a tube structure.

The elongated flexible sheath 106, 206 is a clear or translucent plastic sheath that includes a proximal seal 114, 214 at the proximal end 110, 210 and a distal seal 112, 212 at the distal end 108, 208. In an embodiment, the distal seal 112, 212 includes a barrier 120, 220, such as an absorbent pad or a hot melt plug or both. In an embodiment, the lighting element 102, 202 is also separated from the malleable rod 104, 204 by a barrier 116, 216, 217 such as a hot melt plug or seal. The hot melt plug may be paraffin or a plastic-type material. In an embodiment, the absorbent pad, made from cotton or non-woven fiber, is at the proximal end of the elongated sheath between the lighting element and a terminal seal of the elongated flexible sheath to absorb chemical or liquid from the lighting element 102, 202.

The stylet 100, 200 can include one or more tactile elements 130, 132, 230, 232 configured to enable tactile location of the lighting element and/or the malleable rod. The tactile element 130, 132, 230, 232 in an embodiment is a concentric ring within the sheath, or an etching or a textured area on or around the flexible sheath. The tactile elements may be used, for example, to locate the vial to begin the chemical reaction to produce the light. In another example, the tactile element is used to locate a proximal end of the malleable rod in order to flex or bend the rod to create a handle. In another example, the tactile elements are useful in locating the distal end to bend the distal end for easier insertion into the oral cavity.

The stylet 100, 200 can also include a protective cap 140, 240 that is removable from the distal end and prevents premature activation of the lighting element. The protective cap or sleeve may cover the entire length of the stylet or a portion of the stylet. In an embodiment, the protective cap 140, 240 includes a lubricant 142, 242, to facilitate insertion of the stylet into the trachea. The cap 140, 240 prevents bending and premature activation of the lighting element and can be, for example, dark or colored to minimize exposure of the lighting element to ultraviolet light.

To activate the light, the fragile vial separating the two components is broken. The oxalate is enclosed within a vial 302 (shown in FIG. 3) within the lighting element 102, 202. The activator and oxalate mix to form a light emitting substance that is visible through the transparent or translucent material of the elongated flexible sheath. Typically, one of the components (an activator) is formulated with dimethyl phthalate with hydrogen peroxide, a salt such as sodium salicylate to act as a catalyst, and some tertiarybutyl alcohol to help maintain the peroxide in solution. The other component (an oxalate) typically includes bis(2,4,5-trichlorophenyl-6-carbopentoxyphenyl)oxalate (CPPO) in dibutyl phthalate with a dye. In another embodiment, a chemiluminescent formulation includes an oxalate solution including a dye and an activator solution including polyethylene oxide. In yet another embodiment, the chemiluminescent formulation is phthalate-free, such as is described in Cranor, U.S. Pat. No. 6,126,871 and incorporated herein by reference. Such a chemiluminescent formulation uses a triethyl citrate (TEC)-based activator.

The chemical reaction produces a glowing light which lasts for approximately five (5) minutes. In an embodiment, a peak intensity of light is attained at 20-30 seconds after the initial mixing of the components. In another embodiment, the lighting element 102, 202 not only illuminates, it glows a color, such as blue light, that is visible through green exudate, red blood, or clear/serous or serosanguinous fluid. The lighting element may be formulated to glow a variety of different colors, such as pink, red, yellow, blue, green, aqua, purple, and white. It is contemplated that specific colors may be chosen depending on the lighting requirements and specific application conditions. In addition, the lighting element may be configured for temperature variations. For example, it is contemplated that an embodiment of a lighting element formulation may be suitable for warm weather while another embodiment is suitable for cold weather application or both. In yet another embodiment, the light may appear brighter when properly placed in, for example the trachea, and not glow when placed incorrectly, such as in the esophagus. Such an embodiment minimizes false placement of the stylet.

A method of making a chemiluminescent stylet for use with an endotracheal tube includes extruding or providing an elongated, flexible, polymer sheath and heat-sealing the flexible polymer sheath at a first end. The elongated flexible sheath may be formed from one or more polymers. The lighting element is added by inserting an oxalate vial and vacuum-filling the flexible polymer sheath with an activator. Tactile elements may be introduced in an embodiment by, for example, inserting a concentric ring into the elongated flexible sheath. A malleable rod is then inserted and the proximal end (second end) of the flexible sheath is heat-sealed. The entire stylet is then washed in methanol. In another embodiment, a second malleable rod is inserted into the distal end of the elongated flexible sheath, prior to insertion of the lighting elements.

The malleable rod(s) may be an annealed aluminum rod or a galvanized metal rod. Other tactile elements, such as laser etching may be introduced to the elongated flexible sheath as well. In another embodiment, a second malleable rod is inserted into the distal end of the elongated flexible sheath, prior to insertion of the lighting elements. Also, in another embodiment, as each of the stylet elements are added they are followed by sealing or a hot plug in order to separate the lighting elements from the malleable rod within the elongated flexible sheath.

Advantages to the lighted stylet will be appreciated by those skilled in the art. The stylet is fully enclosed with no batteries, fiber optics, light bulbs, or wiring. Sufficient light is provided to illuminate event the darkest of oral cavities and the light may be positioned directly where needed most. The chemiluminescent lighting element is used once then disposed of, preventing cross-contamination and eliminating the need for sterilization of the stylet.

All patents referred to herein, are incorporated herein by reference, whether or not specifically done so within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A stylet for use with an endotracheal tube, the stylet comprising:
a lighting element;
at least one malleable rod; and
an elongated flexible sheath,
wherein the lighting element and the at least one malleable rod are completely enclosed within the elongated flexible sheath, the elongated flexible sheath having a proximal end and a distal end, and wherein the lighting element is positioned at or near the distal end of the elongated flexible sheath and one of the at least one malleable rod is positioned from proximal to the lighting element to the proximal end of the elongated flexible sheath.

2. The stylet of claim 1 wherein the lighting element is a chemiluminescent lighting element including an activator and an oxalate, wherein the oxalate is enclosed within a glass vial within the lighting element.

3. The stylet of claim 2 wherein the vial of oxalate and the activator are enclosed within a capsule.

4. The stylet of claim 1 wherein the elongated flexible sheath includes a proximal seal at the proximal end and a distal seal at the distal end.

5. The stylet of claim 4 wherein the distal seal includes at least one of an absorbent pad and a hot melt plug.

6. The stylet of claim 1 wherein the lighting element is separated from the at least one malleable rod by a barrier.

7. The stylet of claim 1 wherein the elongated flexible sheath is an outer wall of the lighting element.

8. The stylet of claim 1 wherein the stylet includes one or more tactile elements configured to enable location of the lighting element or the at least one malleable rod.

9. The stylet of claim 8 wherein the tactile element is a concentric ring, an etching, or a textured sheath area.

10. The stylet of claim 1 further comprising a protective cap, the protective cap removable from the distal end and configured to prevent activation of the lighting element.

11. The stylet of claim 10 wherein the protective cap includes a lubricant.

12. The stylet of claim 10 wherein the protective cap is configured to prevent bending of the stylet.

13. The stylet of claim 10 wherein the protective cap is configured to prevent exposure of the stylet to ultraviolet light.

14. The stylet of claim 1 wherein the lighting element has approximately a five (5) minute total effective light output.

15. The stylet of claim 1 wherein an absorbent pad is at the distal end of the elongated sheath between the lighting element and a terminal seal of the elongated flexible sheath.

16. The stylet of claim 15 wherein the absorbent pad is made from cotton or a non-woven polymer.

17. A method of making a chemiluminescent stylet for use with an endotracheal tube comprising:
providing a flexible polymer sheath;
heat-sealing the flexible polymer sheath at a first end;
inserting an oxalate vial;
vacuum-filling the flexible polymer sheath with an activator;
inserting at least one malleable rod; and
heat-sealing a second end of the flexible polymer sheath.

18. The method of claim 17 further including inserting a concentric ring into the flexible polymer sheath.

19. The method of claim 17 wherein the malleable rod is at least one of an annealed aluminum rod and a galvanized metal rod.

20. The method of claim 17 further including sterilizing the sealed flexible sheath.
